(19) United States
(12) Patent Application Publication
(10) Pub. No.: US 2025/0186704 A1
(43) Pub. Date: Jun. 12, 2025

(54) DOSE LOGGING ASSEMBLY WITH ROTATIONAL TRANSMISSION FEATURE

(71) Applicant: Novo Nordisk A/S, Bagsvaerd (DK)

(72) Inventors: Bjoern Juul, Broendby (DK); Jan Harald Preuthun, Broenshoej (DK)

(21) Appl. No.: 18/845,559

(22) PCT Filed: Mar. 2, 2023
(86) PCT No.: PCT/EP2023/055336

§ 371 Date: Sep. 10, 2024

(30) Foreign Application Priority Data
Mar. 10, 2022 (EP) 22161355.7

(51) Int. Cl. A61M 5/315

(52) U.S. Cl. CPC A61M 5/31568; A61M 2205/3327

(57) ABSTRACT

Add-on device comprising a housing, a dose setting assembly and a dose release assembly with a sensor assembly mounted in the housing. The dose setting assembly comprises a gripping member adapted to non-rotationally engage a pen device dose setting member, and an add-on dose setting member rotationally coupled to the housing outer surface as well as to the gripping member allowing the gripping member to be rotated at least 360 degrees. The sensor assembly is non-rotationally coupled to the housing and axially moveable between a proximal position and a distal position relative to the housing to engage and actuate a pen device release member when moved distally.

The present invention generally relates to medical devices for which efficient and reliable detection of expelled dose amounts is relevant.

### BACKGROUND OF THE INVENTION

In the disclosure of the present invention reference is mostly made to drug delivery devices used e.g. in the treatment of diabetes by subcutaneous delivery of insulin, however, this is only an exemplary use of the present invention.

Drug delivery devices for subcutaneous injections have greatly improved the lives of patients who must self-administer drugs and biological agents. Such drug delivery devices may take many forms, including simple disposable devices that are little more than an ampoule with an injection means or they may be durable devices adapted to be used with prefilled cartridges. Regardless of their form and type, they have proven to be great aids in assisting patients to self-administer injectable drugs and biological agents. They also greatly assist care givers in administering injectable medicines to people incapable of performing self-injections. A common type of drug delivery devices allows a user to set a desired dose size for the drug to be delivered. For a typical mechanical device the dose setting means is in the form of a rotatable dose setting or dial member allowing the user to set (or “dial”) the desired dose size which is then subsequently expelled from the device.

Performing the necessary insulin injection at the right time and in the right size is essential for managing diabetes, i.e. compliance with the specified insulin regimen is important. In order to make it possible for medical personnel to determine the effectiveness of a prescribed dosage pattern, diabetes patients are encouraged to keep a log of the size and time of each injection. However, such logs are normally kept in handwritten notebooks, and the logged information may not be easily uploaded to a computer for data processing. Furthermore, as only events, which are noted by the patient, are logged, the notebook system requires that the patient remembers to log each injection, if the logged information is to have any value in the treatment of the patient's disease. A missing or erroneous record in the log results in a misleading picture of the injection history and thus a misleading basis for the medical personnel's decision making with respect to future medication. Accordingly, it may be desirable to automate the logging of injection information from medication delivery systems.

Though some injection devices integrate this monitoring/acquisition mechanism into the device itself, e.g. as disclosed in US 2009/0318865 and WO 2010/052275, most devices of today are without it. The most widely used devices are purely mechanical devices being either durable or prefilled. The latter devices are to be discarded after being emptied and so inexpensive that it is not cost-effective to build-in electronic data acquisition functionality in the device itself. Addressing this problem a number of solutions have been proposed which would help a user to generate, collect and distribute data indicative of the use of a given medical device.

For example, WO 2014/037331 describes in a first embodiment an electronic supplementary device (also named “add-on module” or “add-on device”) adapted to be releasably attached to a drug delivery device of the pen type. The device includes a camera and is configured to perform optical character recognition (OCR) on captured images from a rotating scale drum visible through a dosage window on the drug delivery device, thereby to determine a dose of medicament that has been dialled into the drug delivery device. WO 2014/020008 discloses an electronic supplementary device adapted to be releasably attached to a drug delivery device of the pen type. The device includes a camera and is configured to determine scale drum values based on OCR. To properly determine the size of an expelled dose the supplementary device further comprises additional electromechanical sensor means to determine whether a dose size is set, corrected or delivered. A further external device for a pen device is shown in WO 2014/161952.

WO 2020/176317 discloses a drug delivery device comprising dose logging sensor circuitry incorporated in a dose button. During dose setting the dose button is rotationally locked to a dose-setting screw and thus moves axially therewith in the proximal direction when setting a dose. When the set dose is to be expelled the user moves the button and thus the screw distally to thereby expel the set dose. During distal movement a rotational clutch between the dose button and the screw is released, this allowing the screw to rotate whereas the dose button is held rotationally fixed by the user's finger, whereby relative rotation between the sensor circuitry and the screw allows the size of an expelled dose to be determined.

WO 2019/057911 and WO 2019/162235 disclose an add-on dose logging device for a pen-formed drug delivery device, comprising sensor means adapted to capture an amount of rotation of a magnetic member arranged in the drug delivery device, the amount of rotation of the magnetic member corresponding to the amount of drug expelled from a reservoir by the drug delivery device. During dose setting the sensor means is coupled to and rotates with an outer dose setting member, the two components being rotationally decoupled during dose expelling to prevent transfer of rotation from the dose setting to the sensor means.

The add-on device disclosed in WO 2019/057911 and WO 2019/162235 comprises an electronic sensor module which during dose setting is rotationally coupled to both an external dose setting member and an inner dose setting member of the pen drug delivery device, whereby rotation of the external dose setting member is transferred to the inner dose setting member via the sensor module. However, during dose expelling it is important that the sensor module does not rotate for which reason the add-on device is provided with a mechanism that rotationally decouples the sensor module from the outer dose setting member when the dose release button is actuated to cause expelling of a set dose.

As the decoupling mechanism adds both complexity, costs and bulk to the add-on dose logging device a simplified design would be desirable. Thus, having regard to the above, it is an object of the present invention to provide devices, assemblies and methods allowing efficient, reliable and precise capturing of expelled dose amounts based on the determination of the amount of rotation for a given indicator component or structure.

Further, when the sensor module during dose setting rotates relative to the exterior of the logging device it is difficult to provide a display or other visual communication means on an exterior surface of the device. Correspondingly, it is a further object of the present invention to provide an arrangement in which the sensor module does not rotate during operation, this allowing a display or indicator to be provided cost-effectively. The devices, assemblies and methods may relate to add-on devices adapted to be releasably mounted on a drug delivery device as well as to sensor arrangements formed integrally with a given drug delivery device.

### DISCLOSURE OF THE INVENTION

In the disclosure of the present invention, embodiments and aspects will be described which will address one or more of the above objects or which will address objects apparent from the below disclosure as well as from the description of exemplary embodiments.

As appears from the above description of the add-on device known from WO 2019/057911 and WO 2019/162235, the sensor module is not actually rotationally locked relative to the expelling mechanism during dose expelling but merely prevented from being rotationally influenced from the outside.

The present invention is based on the realization that an arrangement in which the sensor module is rotationally locked to the drug delivery device (but axially moveable) during both dose setting and dose expelling would allow the previous coupling arrangement to be dispensed with, this potentially providing a simplified design.

Thus, in a first general aspect of the invention an add-on device adapted to be releasably mounted on a drug delivery device is provided, the drug delivery device comprising a housing, a drug reservoir or means for receiving a drug reservoir, drug expelling means comprising a rotatable dose setting member allowing a user to set a dose amount of drug to be expelled, a proximally arranged release member actuatable between a proximal position and a distal position, the proximal position allowing a dose amount to be set, the distal position allowing the drug expelling means to expel a set dose, and an indicator adapted to move during expelling of a dose amount, the amount of movement being indicative of the size of the expelled dose amount. The add-on device comprises an add-on dose setting assembly, and an add-on dose release assembly, the add-on dose release assembly comprising a generally tubular add-on housing defining a reference axis, the add-on housing comprising a distal opening adapted to receive the proximal portion of the drug delivery device and being releasably attachable to the drug delivery device housing, the add-on housing comprising an interior space and an outer surface, and a sensor assembly arranged in the interior of the tubular add-on housing and coupled, directly or indirectly, non-rotationally yet axially moveable thereto, the sensor assembly being operatable to detect the amount of rotation of the indicator during expelling of a dose amount. The add-on dose setting assembly comprises a gripping member rotationally arranged in the interior of the add-on housing and adapted to non-rotationally engage the dose setting member, and an add-on dose setting member rotationally coupled to the tubular housing outer surface. The add-on dose setting member is coupled to the gripping member allowing the gripping member to be rotated at least 360 degrees, and the sensor assembly is axially moveable between a proximal position and a distal position relative to the add-on housing to, with the add-on device mounted on the drug delivery device, engage and actuate the release member when moved distally.

By this arrangement the sensor module is rotationally locked to the drug delivery device during both dose setting and dose expelling, this preventing to a high degree rotational movement of the sensor module during dose expelling. Further, such an arrangement allows for simple and cost-effective provision of e.g. an electronically controlled visual indicator should such a feature be desirable.

In exemplary embodiments the add-on housing comprises one or more lateral openings. The gripping member comprises a circumferential outer gear wheel portion having a first diameter and the add-on dose setting member comprises a circumferential inner gear wheel portion having a second larger diameter. The circumferential outer gear wheel portion and the circumferential inner gear wheel portion engage each other, directly or indirectly, corresponding to a lateral opening, whereby rotation of the dose setting member provides rotation of the gripping member and thus the drug delivery device dose setting member.

In an exemplary embodiment the rotational axis of the dose setting member is radially offset from the rotational axis of the gripping member, this allowing the circumferential outer gear wheel portion and the circumferential inner gear wheel portion to engage each other corresponding to a lateral opening to thereby form an internal spur gear.

Alternatively, a transfer gear assembly comprising at least one gear wheel may be arranged corresponding to a lateral opening, the circumferential outer gear wheel portion and the circumferential inner gear wheel portion engaging each other via the transfer gear assembly. The transfer gear assembly may comprise one or more transfer gear wheels just as one or more transfer gear assemblies may be provided.

In an exemplary embodiment the add-on device further comprises a circumferential flexible cam belt arranged through one or more lateral openings (which each may be in the form of a pair of lateral openings next to each other) and comprising (i) an inner toothed surface arranged to engage the gripping member outer gear wheel portion, and (ii) an outer toothed surface arranged to engage the add-on dose setting member inner gear wheel portion. The flexible cam belt is held in engagement with the gripping member in one or more first positions and in contact with the add-on dose setting member in one or more second positions, this providing that rotation of the add-on dose setting member is transferred to the gripping member via the flexible cam belt.

In further exemplary embodiments the add-on dose release assembly comprises a crown member arranged in the interior of the add-on housing. The crown member comprises a plurality of coupling fingers adapted to non-rotationally engage the add-on housing, each finger having an engaged state in engagement with the add-on housing and a non-engaged state. The gripping member comprises at least one bridge portion adapted to pass between the add-on housing and one or more coupling fingers in their non-engaged state, the gripping member being non-rotationally coupled to the add-on dose setting member via the bridge portion(s). A control structure is arranged non-rotationally relative to the add-on dose setting member and in engagement with the coupling fingers to move the coupling fingers between the engaged and non-engaged state to thereby allow the bridge portion(s) to pass the coupling fingers as the control structure and the gripping member rotate relative to the crown member.

The coupling fingers may extend axially and be adapted to move radially between the engaged and the non-engaged state, and the add-on housing may comprise a plurality of receiving structures each adapted to receive a given coupling finger in coupling engagement.

The control structure may comprise a circumferential guide track, and each coupling finger may comprise a free end received in the guide track, this providing that the coupling fingers are moved radially in and out of engagement with the add-on housing when the control structure is rotating together with the add-on dose setting member. The coupling fingers may be in the form of flexible fingers carried by a ring structure.

The control structure may be arranged proximally of and connected to the bridge portion(s) with the guide track facing distally. The control structure may be in the form of a separate member attached to the one or more bridge portions or it may be formed integrally with the gripping member.

In a yet further embodiment the add-on dose setting member comprises a circumferential first toothing, and the gripping member comprises a circumferential second toothing. The add-on device further comprises a transfer ring arranged at an inclined angle relative to a plane perpendicular to the reference axis, the transfer ring comprising (i) a circumferential first transfer toothing in engagement with the add-on dose setting member first toothing, and (ii) a circumferential second transfer toothing in engagement with the gripping member second toothing, whereby rotation of the dose setting member provides rotation of the gripping member via the inclined transfer ring.

The add-on device may be provided as in assembly in combination with a drug delivery device as disclosed above.

In the above-described embodiments the add-on dose release assembly may further comprise a proximally facing user actuatable add-on dose release member coupled to the sensor assembly and moving axially therewith.

In a further aspect of the invention, a unitary drug delivery device with an integrated sensor assembly is provided.

In an exemplary embodiment the unitary drug delivery device comprises a housing defining a reference axis, a drug reservoir or means for receiving a drug reservoir, as well as drug expelling means comprising an inner dose setting member adapted to rotate to set a dose, an inner release member actuatable between a proximal position and a distal position, the proximal position allowing a dose amount to be set, the distal position allowing the drug expelling means to expel a set dose, and an indicator adapted to rotate during expelling of a dose amount, the amount of rotation being indicative of the size of the expelled dose amount. The unitary drug delivery device further comprises a sensor assembly arranged in the interior of the housing and coupled, directly or indirectly, non-rotationally yet axially moveable thereto, the sensor assembly being operatable to detect the amount of rotation of the indicator during expelling of a dose amount. The unitary drug delivery device further comprises a user dose setting member coupled to the housing and adapted to rotate to set a dose, wherein the user dose setting member is rotationally coupled to the inner dose setting member allowing the gripping member to be rotated at least 360 degrees, and the sensor assembly is axially moveable by the user between a proximal position and a distal position relative to the housing to engage and actuate the inner release member when moved distally.

The housing may comprise one or more lateral openings, with the inner dose setting member comprising a circumferential outer gear wheel portion having a first diameter, and the user dose setting member comprising a circumferential inner gear wheel portion having a second larger diameter. The circumferential outer gear wheel portion and the circumferential inner gear wheel portion engage each other, directly or indirectly, corresponding to a lateral opening, whereby rotation of the dose setting member provides rotation of the gripping member.

Alternatively, the unitary drug delivery device further comprises a crown member arranged in the interior of the housing, and a control structure. The crown member comprises a plurality of coupling fingers adapted to non-rotationally engage the housing, each finger having an engaged state in engagement with the add-on housing and a non-engaged state, and the inner dose setting member comprises at least one bridge portion adapted to pass between the housing and one or more coupling fingers in their non-engaged state, the inner dose setting member being non-rotationally coupled to the user dose setting member via the bridge portion(s). The control structure is coupled non-rotationally to the user dose setting member and in engagement with the coupling fingers to control the coupling fingers between the engaged and non-engaged state to thereby allow the bridge portion(s) to pass the coupling fingers as the control structure and the inner dose setting member rotate relative to the crown member.

The unitary drug delivery device may be further modified corresponding to the exemplary embodiments of an add-on sensor device as described above.

In specific embodiments the indicator comprises a plurality of dipole magnets, and the sensor means comprises a plurality of magnetometers arranged non-rotational relative to the housing in a mounted state and adapted to determine magnetic field values from the plurality of dipole magnets, and processor means configured to determine on the basis of measured values from the plurality of magnetometers a rotational position and/or a rotational movement of the indicator.

As used herein, the term drug is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described sensor assembly could also be used to create logs for other types of drugs, e.g. growth hormone.

### BRIEF DESCRIPTION OF THE DRAWINGS

In the following embodiments of the invention will be described with reference to the drawings, wherein

FIG. 1A shows a pen device,

FIG. 1B shows the pen device of FIG. 1A with the pen cap removed,

FIG. 2 shows in an exploded view the components of the pen device of FIG. 1A,

FIGS. 3A and 3B show in sectional views an expelling mechanism in two states,

FIGS. 4A and 4B show a schematic representation of an add-on device and a drug delivery device,

FIGS. 5A and 5B respectively show an exemplary drug delivery device and a corresponding add-on device adapted to be mounted thereon as shown in FIG. 4B,

FIG. 6 shows a schematic representation of a system comprising 3 concentric tubular members,

FIG. 7 shows a schematic of an inner spur gear connection between the inner and outer members of FIG. 6,

FIGS. 8A and 8B show in an exploded view the components of a first embodiment of an add-on dose logging device comprising an inner spur gear connection,

FIG. 9 shows an electronic sensor module mounted in a dose release member,

FIG. 10 shows in a partial cut-away representation the add-on device of FIG. 8A without the sensor module,

FIG. 11 shows in a partial cut-away representation the add-on device of FIG. 8A with the sensor module,

FIG. 12 shows the add-on device of FIG. 8A in a cross-sectional view,

FIG. 13 shows the add-on device of FIG. 8A in an assembled state in combination with a drug delivery device,

FIG. 14 shows a schematic representation of an alternative gear connection between the inner and outer members of FIG. 6,

FIG. 15 shows a schematic representation of a second embodiment of transfer connection between the inner and outer members of FIG. 6,

FIGS. 16A and 16B show in an exploded view the components of a third embodiment of an add-on dose logging device comprising a dynamic transfer member,

FIG. 17 shows components of the add-on dose logging device of FIG. 16A in an assembled state,

FIG. 18 shows a guide track member of the add-on dose logging device of FIG. 16A,

FIG. 19 shows in a partial cut-away representation the add-on device of FIG. 16A, and

FIG. 20 shows an alternative configuration for a dynamic transfer member.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

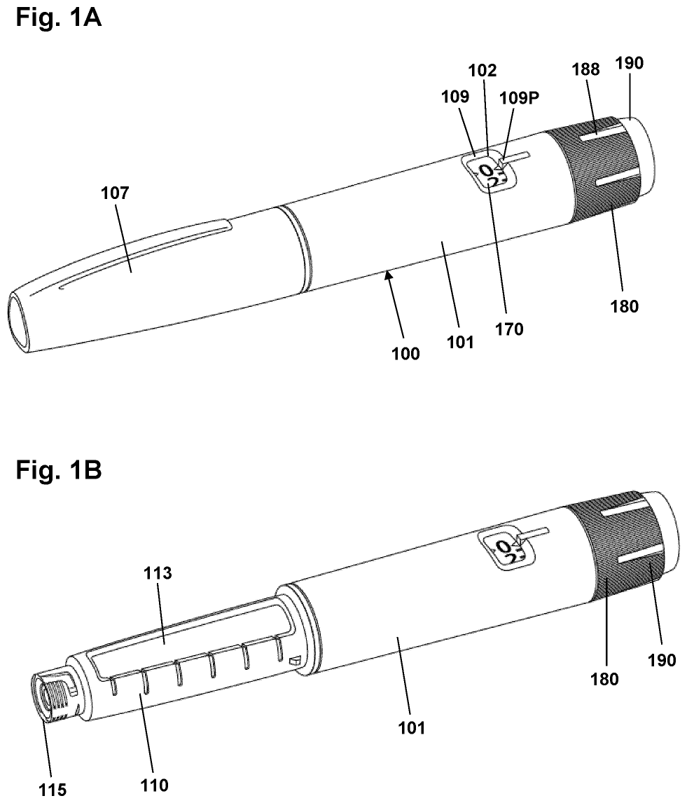

Before turning to embodiments of the present invention per se, an example of a prefilled drug delivery will be described, such a device providing the basis for the exemplary embodiments of the present invention. Although the pen-formed drug delivery device 100 shown in FIGS. 1-3 may represent a “generic” drug delivery device, the actually shown device is a FlexTouch® prefilled drug delivery pen as manufactured and sold by Novo Nordisk A/S, Bagsværd, Denmark.

The pen device 100 comprises a cap part 107 and a main part having a proximal body or drive assembly portion with a housing 101 in which a drug expelling mechanism is arranged or integrated, and a distal cartridge holder portion in which a drug-filled transparent cartridge 113 with a distal needle-penetrable septum is arranged and retained in place by a non-removable cartridge holder attached to the proximal portion, the cartridge holder having openings allowing a portion of the cartridge to be inspected as well as distal coupling means 115 allowing a needle assembly to be releasably mounted. The cartridge is provided with a piston driven by a piston rod forming part of the expelling mechanism and may for example contain an insulin, GLP-1 or growth hormone formulation. A proximal-most rotatable dose setting member 180 with a number of axially oriented grooves 188 serves to manually set a desired dose of drug shown in display window 102 and which can then be expelled when the button 190 is actuated. As will be apparent from the below description, the shown axially oriented grooves 188 may be termed “drive grooves”. The dose setting member 180 has a generally cylindrical outer surface 181 (i.e. the dose setting member may be slightly tapered) which in the shown embodiment is textured by comprising a plurality of axially oriented fine grooves to improve finger grip during dose setting. The window is in the form of an opening in the housing surrounded by a chamfered edge portion 109 and a dose pointer 109P, the window allowing a portion of a helically rotatable indicator member 170 (scale drum) to be observed. Depending on the type of expelling mechanism embodied in the drug delivery device, the expelling mechanism may comprise a spring as in the shown embodiment which is strained during dose setting and then released to drive the piston rod when the release button is actuated. Alternatively the expelling mechanism may be fully manual in which case the dose member and the actuation button move proximally during dose setting corresponding to the set dose size, and then is moved distally by the user to expel the set dose, e.g. as in a FlexPen® manufactured and sold by Novo Nordisk A/S.

Although FIG. 1 shows a drug delivery device of the prefilled type, i.e. it is supplied with a premounted cartridge and is to be discarded when the cartridge has been emptied, in alternative embodiments the drug delivery device may be designed to allow a loaded cartridge to be replaced, e.g. in the form of a “rear-loaded” drug delivery device in which the cartridge holder is adapted to be removed from the device main portion, or alternatively in the form of a “frontloaded” device in which a cartridge is inserted through a distal opening in the cartridge holder which is non-removable attached to the main part of the device.

As the invention relates to electronic circuitry adapted to interact with a drug delivery device, an exemplary embodiment of such a device will be described for better understanding of the invention.

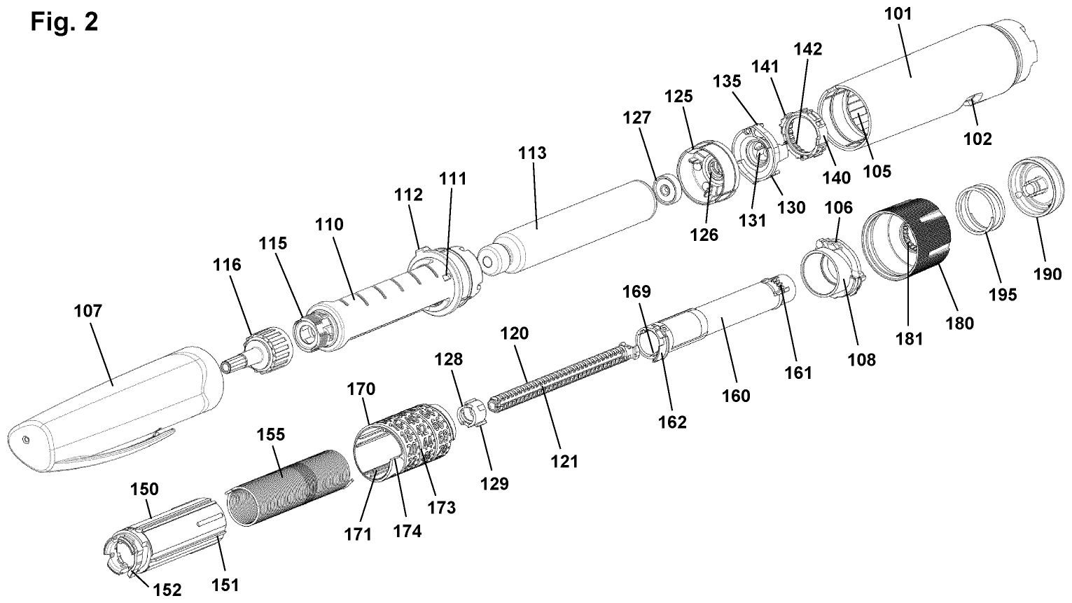

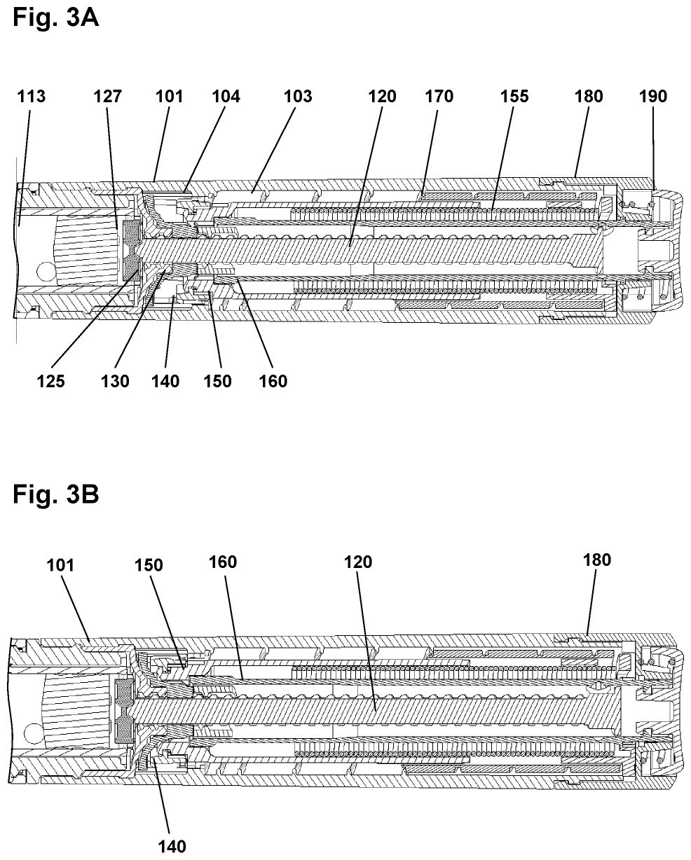

FIG. 2 shows an exploded view of the pen-formed drug delivery device 100 shown in FIG. 1. More specifically, the pen comprises a tubular housing 101 with a window opening 102 and onto which a cartridge holder 110 is fixedly mounted, a drug-filled cartridge 113 being arranged in the cartridge holder. The cartridge holder is provided with distal coupling means 115 allowing a needle assembly 116 to be releasable mounted, proximal coupling means in the form of two opposed protrusions 111 allowing a cap 107 to be releasable mounted covering the cartridge holder and a mounted needle assembly, as well as a protrusion 112 preventing the pen from rolling on e.g. a tabletop. In the housing distal end a nut element 125 is fixedly mounted, the nut element comprising a central threaded bore 126, and in the housing proximal end a spring base member 108 with a central opening is fixedly mounted. A drive system comprises a threaded piston rod 120 having two opposed longitudinal grooves and being received in the nut element threaded bore, a ring-formed piston rod drive element 130 rotationally arranged in the housing, and a ring-formed clutch element 140 which is in rotational engagement with the drive element (see below), the engagement allowing axial movement of the clutch element. The clutch element is provided with outer spline elements 141 adapted to engage corresponding splines 104 (see FIG. 3B) on the housing inner surface, this allowing the clutch element to be moved between a rotationally locked proximal position, in which the splines are in engagement, and a rotationally free distal position in which the splines are out of engagement. As just mentioned, in both positions the clutch element is rotationally locked to the drive element. The drive element comprises a central bore with two opposed protrusions 131 in engagement with the grooves on the piston rod whereby rotation of the drive element results in rotation and thereby distal axial movement of the piston rod due to the threaded engagement between the piston rod and the nut element. The drive element further comprises a pair of opposed circumferentially extending flexible ratchet arms 135 adapted to engage corresponding ratchet teeth 105 arranged on the housing inner surface. The drive element and the clutch element comprise cooperating coupling structures rotationally locking them together but allowing the clutch element to be moved axially, this allowing the clutch element to be moved axially to its distal position in which it is allowed to rotate, thereby transmitting rotational movement from the dial system (see below) to the drive system. The interaction between the clutch element, the drive element and the housing will be shown and described in greater detail with reference to FIGS. 3A and 3B.

On the piston rod an end-of-content (EOC) member 128 is threadedly mounted and on the distal end a washer 127 is rotationally mounted. The EOC member comprises a pair of opposed radial projections 129 for engagement with the reset tube (see below).

The dial system comprises a ratchet tube 150, a reset tube 160, a scale drum 170 with an outer helically arranged pattern forming a row of dose indicia, a user-operated dial member 180 for setting a dose of drug to be expelled, a release button 190 and a torque spring 155 (see FIG. 3). The dial member is provided with a circumferential inner teeth structure 181 engaging a number of corresponding outer teeth 161 arranged on the reset tube, this providing a dial coupling which is in an engaged state when the reset tube is in a proximal position during dose setting and in a disengaged state when the reset tube is moved distally during expelling of a dose. The reset tube is mounted axially locked inside the ratchet tube but is allowed to rotate a few degrees (see below). The reset tube comprises on its inner surface two opposed longitudinal grooves 169 adapted to engage the radial projections 129 of the EOC member, whereby the EOC can be rotated by the reset tube but is allowed to move axially. The clutch element is mounted axially locked on the outer distal end portion of the ratchet tube 150, this providing that the ratchet tube can be moved axially in and out of rotational engagement with the housing via the clutch element. The dial member 180 is mounted axially locked but rotationally free on the housing proximal end, the dial ring being under normal operation rotationally locked to the reset tube (see below), whereby rotation of the dial ring results in a corresponding rotation of the reset tube 160 and thereby the ratchet tube. The release button 190 is axially locked to the reset tube but is free to rotate. A return spring 195 provides a proximally directed force on the button and the thereto mounted reset tube. The scale drum 170 is arranged in the circumferential space between the ratchet tube and the housing, the drum being rotationally locked to the ratchet tube via cooperating longitudinal splines 151, 171 and being in rotational threaded engagement with the inner surface of the housing via cooperating thread structures 103, 173, whereby the row of numerals passes the window opening 102 in the housing when the drum is rotated relative to the housing by the ratchet tube. The torque spring is arranged in the circumferential space between the ratchet tube and the reset tube and is at its proximal end secured to the spring base member 108 and at its distal end to the ratchet tube, whereby the spring is strained when the ratchet tube is rotated relative to the housing by rotation of the dial member. A ratchet mechanism with a flexible ratchet arm 152 is provided between the ratchet tube and the clutch element, the latter being provided with an inner circumferential teeth structures 142, each tooth providing a ratchet stop such that the ratchet tube is held in the position to which it is rotated by a user via the reset tube when a dose is set. In order to allow a set dose to be reduced a ratchet release mechanism 162 is provided on the reset tube and acting on the ratchet tube, this allowing a set dose to be reduced by one or more ratchet increments by turning the dial member in the opposite direction, the release mechanism being actuated when the reset tube is rotated the above-described few degrees relative to the ratchet tube.

Having described the different components of the expelling mechanism and their functional relationship, operation of the mechanism will be described next with reference mainly to FIGS. 3A and 3B.

The pen mechanism can be considered as two interacting systems, a dose system and a dial system, this as described above. During dose setting the dial mechanism rotates and the torsion spring is loaded. The dose mechanism is locked to the housing and cannot move. When the push button is pushed down, the dose mechanism is released from the housing and due to the engagement to the dial system the torsion spring will now rotate back the dial system to the starting point and rotate the dose system along with it.

The central part of the dose mechanism is the piston rod 120, the actual displacement of the plunger being performed by the piston rod. During dose delivery, the piston rod is rotated by the drive element 130 and due to the threaded interaction with the nut element 125 which is fixed to the housing, the piston rod moves forward in the distal direction. Between the rubber piston and the piston rod, the piston washer 127 is placed which serves as an axial bearing for the rotating piston rod and evens out the pressure on the rubber piston. As the piston rod has a non-circular cross section where the piston rod drive element engages with the piston rod, the drive element is locked rotationally to the piston rod, but free to move along the piston rod axis. Consequently, rotation of the drive element results in a linear forwards movement of the piston. The drive element is provided with small ratchet arms 134 which prevent the drive element from rotating clockwise (seen from the push button end). Due to the engagement with the drive element, the piston rod can thus only move forwards. During dose delivery, the drive element rotates anti-clockwise and the ratchet arms 135 provide the user with small clicks due to the engagement with the ratchet teeth 105, e.g. one click per unit of insulin expelled. Turning to the dial system, the dose is set and reset by turning the dial member 180. When turning the dial, the reset tube 160, the EOC member 128, the ratchet tube 150 and the scale drum 170 all turn with it due to the dial coupling being in the engaged state. As the ratchet tube is connected to the distal end of the torque spring 155, the spring is loaded. During dose setting, the arm 152 of the ratchet performs a dial click for each unit dialled due to the interaction with the inner teeth structure 142 of the clutch element. In the shown embodiment the clutch element is provided with 24 ratchet stops providing 24 clicks (increments) for a full 360 degrees rotation relative to the housing. The spring is preloaded during assembly which enables the mechanism to deliver both small and large doses within an acceptable speed interval. As the scale drum is rotationally engaged with the ratchet tube, but movable in the axial direction and the scale drum is in threaded engagement with the housing, the scale drum will move in a helical pattern when the dial system is turned, the number corresponding to the set dose being shown in the housing window 102.

The ratchet 152, 142 between the ratchet tube and the clutch element 140 prevents the spring from turning back the parts. During resetting, the reset tube moves the ratchet arm 152, thereby releasing the ratchet click by click, one click corresponding to 1 IU in the described embodiment. More specifically, when the dial member is turned clockwise, the reset tube simply rotates the ratchet tube allowing the arm of the ratchet to freely interact with the teeth structures 142 in the clutch element. When the dial member is turned counter-clockwise, the reset tube interacts directly with the ratchet click arm forcing the click arm towards the centre of the pen away from the teeth in the clutch, thus allowing the click arm on the ratchet to move “one click” backwards due to torque caused by the loaded spring.

To deliver a set dose, the push button 190 is pushed in the distal direction by the user as shown in FIG. 3B. The dial coupling 161, 181 disengages and the reset tube 160 decouples from the dial member and subsequently the clutch element 140 disengages the housing splines 104. Now the dial mechanism returns to “zero” together with the drive element 130, this leading to a dose of drug being expelled. It is possible to stop and start a dose at any time by releasing or pushing the push button at any time during drug delivery. A dose of less than 5 U normally cannot be paused since the rubber piston is compressed very quickly leading to a compression of the rubber piston and subsequently delivery of insulin when the piston returns to the original dimensions.

The EOC feature prevents the user from setting a larger dose than left in the cartridge. The EOC member 128 is rotationally locked to the reset tube, which makes the EOC member rotate during dose setting, resetting and dose delivery, during which it can be moved axially back and forth following the thread of the piston rod. When it reaches the proximal end of the piston rod a stop is provided, this preventing all the connected parts, including the dial member, from being rotated further in the dose setting direction, i.e. the now set dose corresponds to the remaining drug content in the cartridge.

The scale drum 170 is provided with a distal stop surface 174 adapted to engage a corresponding stop surface on the housing inner surface, this providing a maximum dose stop for the scale drum preventing all the connected parts, including the dial member, from being rotated further in the dose setting direction. In the shown embodiment the maximum dose is set to 80 IU. Correspondingly, the scale drum is provided with a proximal stop surface adapted to engage a corresponding stop surface on the spring base member, this preventing all the connected parts, including the dial member, from being rotated further in the dose expelling direction, thereby providing a “zero” stop for the entire expelling mechanism.

To prevent accidental overdosage in case something should fail in the dialling mechanism allowing the scale drum to move beyond its zero-position, the EOC member serves to provide a security system. More specifically, in an initial state with a full cartridge the EOC member is positioned in a distal-most axial position in contact with the drive element. After a given dose has been expelled the EOC member will again be positioned in contact with the drive element. Correspondingly, the EOC member will lock against the drive element in case the mechanism tries to deliver a dose beyond the zero-position. Due to tolerances and flexibility of the different parts of the mechanism the EOC will travel a short distance allowing a small “overdose” of drug to be expelled, e.g. 3-5 IU of insulin.

The expelling mechanism further comprises an end-of-dose (EOD) click feature providing a distinct feedback sound at the end of an expelled dose informing the user that the full amount of drug has been expelled. More specifically, the EOD function is made by the interaction between the spring base and the scale drum. When the scale drum returns to zero, a small click-arm 106 on the spring base is forced backwards by the progressing scale drum. Just before “zero” the arm is released, and the arm hits a countersunk surface on the scale drum.

The shown mechanism is further provided with a torque limiter in order to protect the mechanism from overload applied by the user via the dial member. This feature is provided by the interface between the dial member and the reset tube which as described above are rotationally locked to each other. More specifically, the dial member is provided with circumferential inner teeth structure 181 engaging a number of corresponding outer teeth 161, the latter being arranged on a flexible carrier portion of the reset tube. The reset tube teeth are designed to transmit a torque of a given specified maximum size, e.g. 150-300 Nmm, above which the flexible carrier portion and the teeth will bend inwards and make the dial member turn without rotating the rest of the dial mechanism. Thus, the mechanism inside the pen cannot be stressed at a higher load than the torque limiter transmits through the teeth.

Having described the working principles of a mechanical drug delivery device, embodiments of the present invention will be described.

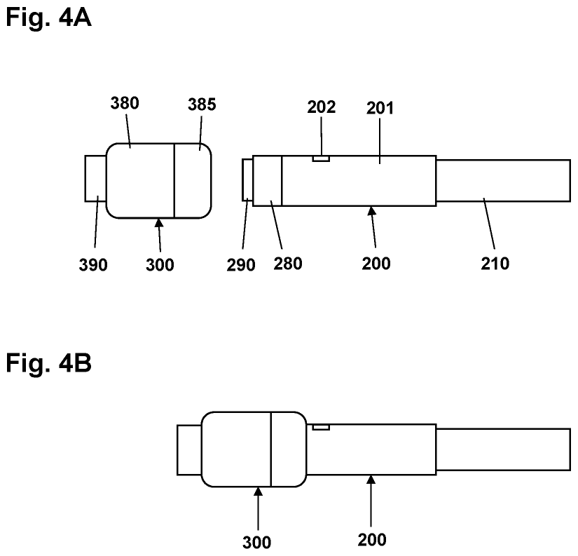

FIGS. 4A and 4B show a schematic representation of a first assembly of a pre-filled pen-formed drug delivery device 200 and a therefor adapted add-on dose logging device 300. The add-on device is adapted to be mounted on the proximal end portion of the pen device housing and is provided with dose setting and dose release means 380 covering the corresponding means on the pen device in a mounted state as shown in FIG. 4B. In the shown embodiment the add-on device comprises a coupling portion 385 adapted to be mounted axially and rotationally locked on the drug delivery housing. The add-on device comprises a rotatable dose setting member 380 which during dose setting is directly or indirectly coupled to the pen dose setting member 280 such that rotational movement of the add-on dose setting member in either direction is transferred to the pen dose setting member. In order to reduce influences from the outside during dose expelling and dose size determination, the outer add-on dose setting member 380 may be rotationally decoupled from the pen dose setting member 280 during dose release and expelling. The add-on device further comprises a dose release member 390 which can be moved distally to thereby actuate the pen release member 290.

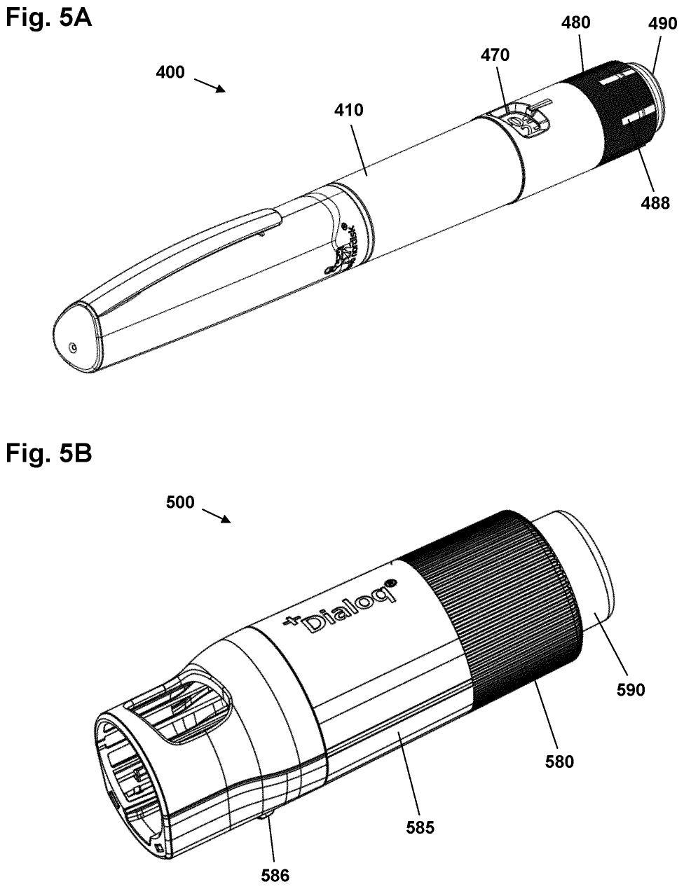

Turning to FIGS. 5A and 5B a specific prior art embodiment of an add-on dose logging device 500 adapted to be mounted on a pen-formed drug delivery device 400 is shown, the pen device essentially corresponding to a FlexTouch® prefilled drug delivery pen with the shown add-on dose logging device being branded as “Dialoq®”. The add-on device 500 essentially corresponds to the above-described add-on dose logging device 300 and thus comprises a housing portion 585 adapted to be mounted axially and rotationally locked on the drug delivery housing via releasable coupling means 586, the housing being provided with a window 587 allowing the pen scale drum 470 to be observed during dose setting. The add-on device comprises a rotatable dose setting member 580 which during dose setting is coupled to the pen dose setting member 480 such that rotational movement of the add-on dose setting member in either direction is transferred to the pen dose setting member via coupling grooves 488. The add-on device further comprises a dose release member 590 which can be moved distally to thereby actuate the pen release member 490.

In order to determine the size of an expelled dose amount of drug, the pen device may be provided with magnetic identifiers adapted to rotate during dose expelling and the add-on device may correspondingly be provided with sensor circuitry allowing the amount of rotation to be captured and thereby the expelled dose size to be determined. A number of embodiments based on this concept is disclosed and described in detail in WO 2019/162235 which is hereby incorporated by reference.

In summary, the add-on device disclosed in WO 2019/162235 comprises an outer assembly being releasably attachable to the drug delivery device housing, and an inner assembly. The outer assembly comprises an add-on dose setting member 580, and an add-on release member 590 axially moveable relative to the add-on dose setting member between a dose setting state and a dose expelling state. The inner assembly comprises an inner dose setting member adapted to engage the dose setting member 480, sensor means adapted to detect the amount of rotation of the indicator during expelling of a dose amount, and an actuator coupled to the add-on release member and being axially moveable between a proximal position and a distal position relative to the inner dose setting member, the actuator being adapted to engage and actuate the pen device release member 490 when moved distally. The sensor circuitry, e.g. in the form of an electronic module, may form part of the actuator (and thus move axially therewith) and be coupled non-rotationally to the inner dose setting member to prevent rotation during dose expelling. The sensor circuitry will typically be activated from a sleep state when the user actuates and axially moves the add-on release member 590.

As the decoupling mechanism adds both complexity, costs and bulk to the add-on dose logging device a simplified design would be desirable. As also indicated above, the present invention is based on the realization that an arrangement in which the sensor module is rotationally locked to the drug delivery device (but axially moveable) during both dose setting and dose expelling would allow the coupling arrangement to be dispensed with, this potentially providing a simplified design and/or more compact design.

Correspondingly, in the following exemplary embodiments are provided ensuring that a sensor module is rotationally locked to a drug delivery device/assembly (but axially moveable) during both dose setting and dose expelling.

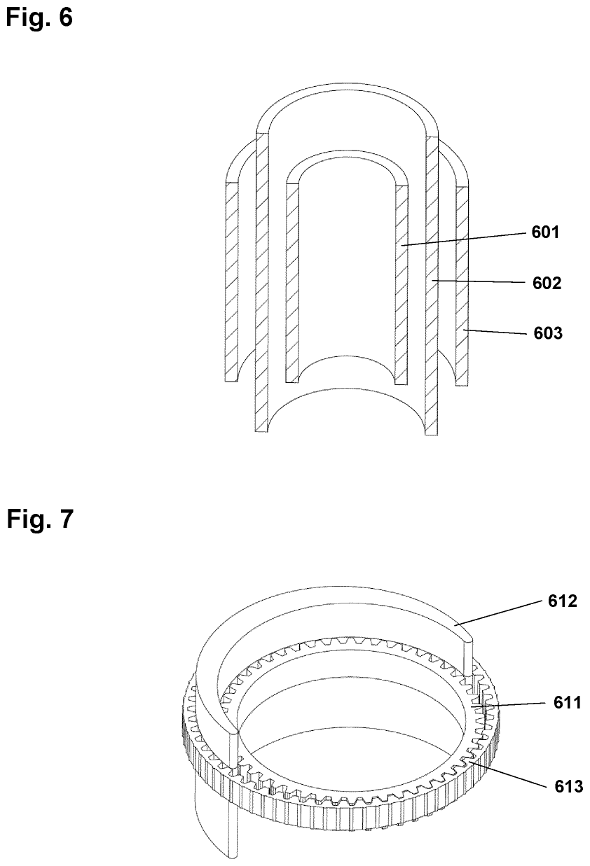

In a mechanical system, components are required to reference to each other, hereby meaning that components orientation and/or position in some manner follows other components in the system. An add-on device of the type disclosed in WO 2019/162235 essentially comprises a housing non-moveable mounted on the pen housing, an inner dose setting coupling member rotationally coupled to the pen dose setting member, and an outer rotatable dose setting member allowing a user to set a dose, i.e. rotation of the outer dose setting member is transferred to the inner dose setting member. Such a system can be compared with three tubes 601, 602, 603 of increasing diameter assembled one into the other as illustrated in FIG. 6.

The challenge in the system illustrated in FIG. 6 is thus to reference the outer layer to the inner layer through the intermediate layer, in other words to ensure that the tube rotates when the outer tube is rotated, this without involving the electronic module. At a first glance this would require the inner tube to have a rigid connection to the outer tube through the intermediate tube, however as the outer tube is to be rotated more than 360 degrees a circumferential slit or opening would be required in the intermediate layer, this effectively dividing the intermediate tube in two.

In a first exemplary embodiment a rotational coupling mechanism is provided between the inner and outer dose setting members through an opening in the add-on housing (corresponding to the intermediate layer in FIG. 6) allowing both the inner and outer dose setting member to be rotated at least 360 degrees.

More specifically and as schematically shown in FIG. 7, an internal spur gear connection is provided between the inner and outer dose setting members 611, 613, the outer larger diameter dose setting member comprising an inner circumferential gear toothing engaging an outer circumferential gear toothing on the smaller diameter inner dose setting member. The outer dose setting member is co-axially and rotationally arranged on a cylindrical portion of the add-on housing 612, and the inner dose setting member is arranged rotationally but axially offset in the interior of the tubular housing, the two gears meshing with each other through an opening/slot in the add-on housing. As appears the offset arrangement of the inner dose setting member allows the add-on housing to distend both proximally and distally relative to the geared connection between the two dose setting members. In this way the distal housing portion can be used to non-rotationally engage the pen body housing and the proximal housing portion can be used to house the electronic sensor unit axially moveable but non-rotationally coupled to the housing, this providing a non-rotational coupling between the sensor module and the pen housing. As the inner dose setting member is adapted to engage the pen dose setting member and thus has to be arranged co-axially therewith with the add-on device mounted on the pen device, it follows that the add-on housing has to be mounted axially offset on the pen housing.

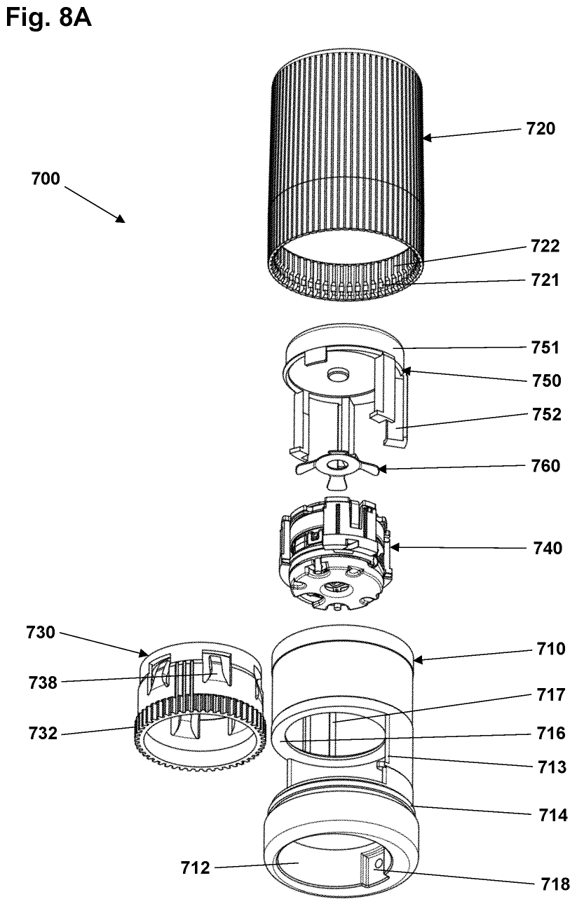

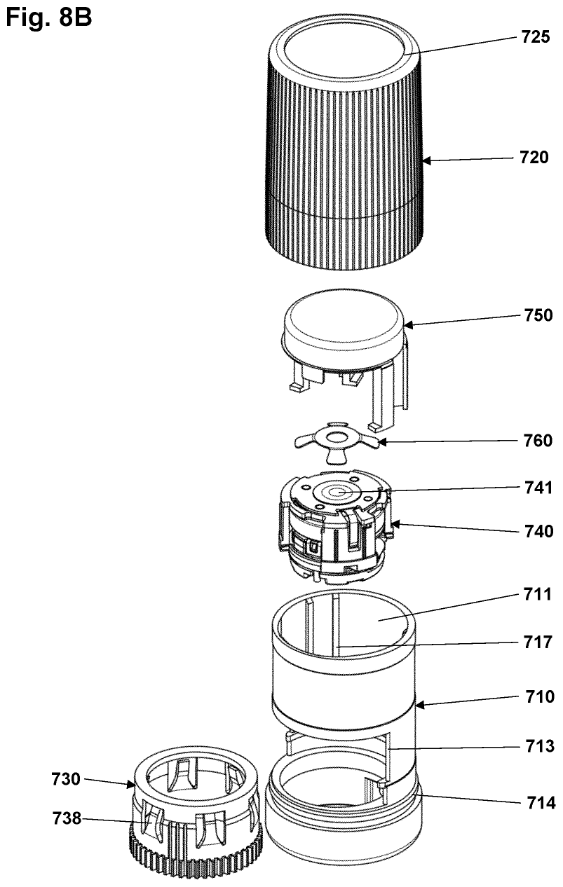

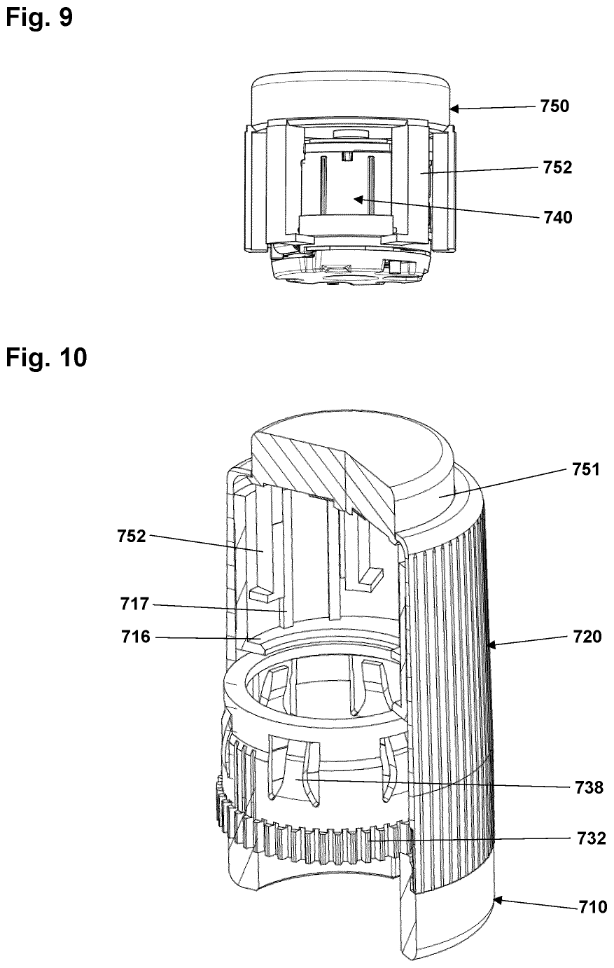

Turning to FIGS. 8A, 8B and 9-13 a specific embodiment of an add-on device 700 comprising an internal spur gear connection between the inner and outer dose setting members will be described.

More specifically, the add-on device comprises a tubular housing member 710 with a proximal opening 711, a distal opening 712 adapted to receive the proximal end of a pen device as well as a lateral opening 713, an outer tubular dose setting (dial) member 720, an inner tubular dose setting member 730 adapted to engage (grip) the pen dose setting member, a sensor module 740 with a proximal switch 741, a button member 750 adapted to house the sensor module, a switch return spring 760, and (optionally) a button member/sensor module return spring.

The housing member 710 further comprises an outer circumferential ridge structure 714 at the distal end adapted to engage the outer dose setting member 720, inner circumferential receiving means adapted to engage the inner dose setting member 730, as well as inner stop 716 and axial guide means 717 adapted to engage the button member 750. The housing member further comprises releasable locking means 718 allowing the add-on device to be mounted axially as well as rotationally locked on the pen housing.

The tubular outer dose setting member 720 comprises a generally smooth inner surface adapted to rotate on the housing outer tubular surface, a distal inner circumferential groove 721 adapted to rotationally engage the housing outer ridge structure, a distal inner gear toothing 722 adapted to engage the inner dose setting member 730, and a proximal inwardly protruding flange 725 adapted to provide an axial stop for the button member 750.

The tubular inner dose setting member 730 comprises a distal outer gear toothing 732 adapted to engage the inner gear toothing on the outer dose setting member, and a proximal portion with a number of inwardly protruding flexible coupling arms 738 adapted to non-rotationally engage corresponding coupling structures on the pen dose setting member. The inner dose setting member is adapted to be mounted axially locked but rotationally free in the housing member 710. As the inner dose setting member is adapted to grip the pen dose setting member it could also be termed a gripping member.

The button member 750 comprises a proximal generally flat button portion 751 and a number of distally protruding rib structures 752 providing a cage structure adapted to receive and hold the sensor module rotationally locked as shown in FIG. 10. The ribs are adapted to non-rotationally engage the housing 710, the button member being axially moveable between a distal stop provided by the housing and a proximal stop provided by the outer dose setting member flange 725.

The sensor module 740 has a generally cylindrical configuration and is adapted to be mounted non-rotationally in the button cage structure with a small axial play, this allowing the switch return spring 760 to be arranged between the switch and the flat button portion to ensure that the sensor module is returned to its distal position in the cage structure after actuation. In a situation of use the distal surface of the sensor module is adapted to engage the pen release button, this allowing the button member to actuate the sensor module switch (see below). The details of the sensor circuitry housed in the sensor module is not relevant in the context of the present invention but is described in detail in e.g. WO 2019/162235.

A button member return spring (not shown) may be arranged between the button member and a housing spring support surface, this assuring that the button member is biased towards its proximal-most position. In case no return spring is provided the button member will be able to move freely axially, however, when mounted on the pen device it will essentially be held axially in place between the pen release button and the button member proximal stop 725.

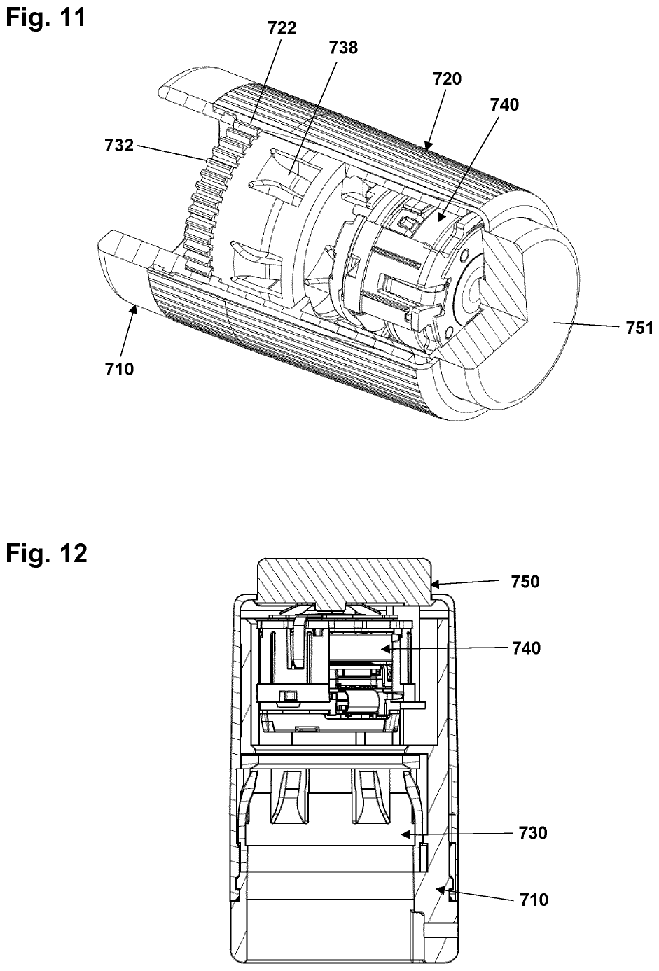

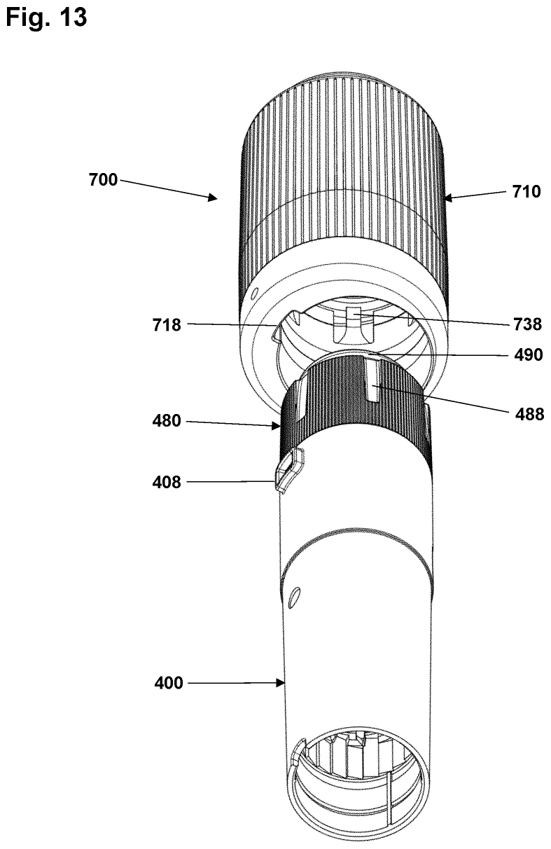

FIGS. 11 and 12 show the inner dose setting member 730 arranged in the housing member 710 and in geared engagement with the outer dose setting member 720, as well as the button member 750 arranged rotationally locked in the housing member. In FIG. 12 the sensor module 740 is shown arranged in the button member.

FIG. 12 shows the assembled add-on device in cross-section which reveals the axially offset arrangement of the sensor module inside the housing. In the shown embodiment a narrow gap is provided between the sensor module and the housing which would allow one or more small diameter return springs to be arranged in the gap.

In a situation of use, see FIG. 13, the user mounts the add-on device 700 on the proximal end of a corresponding pen drug delivery device 400, the add-on releasable locking means 718 engaging a corresponding locking knob 408 on the pen housing 410, whereby the pen dose setting member 480 is received in the inner dose setting member 730. Depending on the design of the latter two members they may become rotationally locked during the mounting operation or, as in the present embodiment, subsequently when the user starts to set a dose and the flexible coupling arms 738 non-rotationally engage corresponding coupling grooves 488 on the pen dose setting member.

To set a dose the user rotates the outer add-on dose setting member 720, and thereby also the inner dose setting member, until the desired dose size is shown in the pen display window. During initial rotation of the inner dose setting member the flexible coupling arms 738 will slide on the pen dose setting member until the arms non-rotationally engage the corresponding axially oriented coupling grooves 488 on the pen dose setting member. As appears, due to the different diameters of the two gear rings a slight gearing is provided between the outer and inner dose setting members which in most cases will not be apparent to the user.

When a desired dose is set the user actuates dose release button 740 this moving the sensor module into engagement with the pen release button. The latter is biased towards its proximal position by a pen button spring, the switch return spring 750 being dimensioned to allow the sensor switch to be actuated and thus turn on the sensor module before the pen release button starts to move distally and subsequently releases the spring-loaded expelling mechanism. During dose expelling the sensor module will determine the amount of rotation of an indicator element, e.g. a magnet as in the present example, and thereby the expelled dose.

When a set dose is expelled, or the user desires to pause injection, the user releases pressure on the dose release button, the button return spring thereby returning the button member with the sensor module to its initial position.

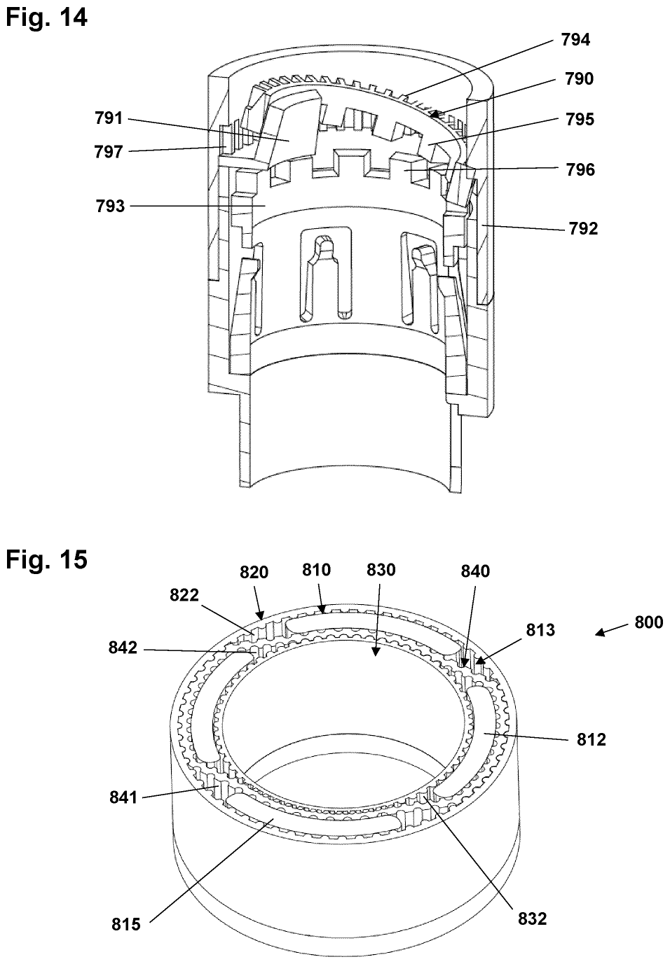

In FIG. 14 is shown an alternative configuration of the first embodiment in which an additional tilted transmission wheel 790 supported by an inner housing support 791 allows the inner and outer dose setting members 793, 792 to be arranged coaxially. The transmission wheel comprises an outer circumferential gear toothing 794 in engagement with the outer dose setting member inner circumferential gear toothing 797, as well as a distally facing circumferential angular gear toothing 795 in engagement with a proximally facing circumferential angular gear toothing 796 on the inner dose setting member. In such a design the housing support 791 would form a structure that could support the electronic module.

With reference to FIG. 15 a second exemplary embodiment of a coupling mechanism 800 allowing an inner and an outer dose setting member to rotate together will be described. Just as in the first exemplary embodiment the concept is to provide a connection through the housing wall at a level distally of the sensor module.

Instead of an internal spur gear arrangement comprising an offset inner dose setting member, FIG. 15 schematically shows a solution in which rotational coupling between an outer dose setting member 820 and an inner dose setting member 830 is provided by a double-sided flexible cambelt 840 comprising an outer and an inner toothing 841, 842 and which is threaded through an opposed pair of openings in an add-on housing member 810.

More specifically, the housing member 810 comprises an opposed pair of openings, each pair comprising two openings 813 having a portion of wall 812 therebetween providing an outer cambelt support surface as well as an inner support surface for the inner dose setting member.

The remaining wall portions 815 provide an inner cambelt support surface as well as an outer support surface for the outer dose setting member.

The outer dose setting member 820 comprises a circumferential inner toothing 822 adapted to engage the outer toothing 841 on the cambelt and the inner dose setting member 830 comprises a circumferential outer toothing 832 adapted to engage the cambelt inner toothing 842.

As shown in FIG. 15 in an assembled state the portion of the cambelt positioned outside the housing is in toothed engagement with the inner toothed surface of the outer dose setting member and the portion of the cambelt positioned inside the housing is in toothed engagement with the outer toothed surface of the inner dose setting member.

As appears, when compared to the first exemplary embodiment comprising an internal spur gear coupling and providing an axially asymmetric design, the cambelt design provides an axially symmetrical design. Otherwise, the cambelt coupling can be integrated in a given add-on dose logging device in essentially the same was as described for the first exemplary embodiment. This said, an internal spur gear arrangement could be provided in an exteriorly symmetrical design.

In an alternative configuration (not shown) the cambelt is arranged through a single pair of lateral openings with the cambelt inner toothing thereby engaging a larger portion of the inner dose setting member outer toothing.

In a further alternative configuration (not shown) the cambelt is replaced with one or more transfer gear assemblies, each assembly comprising one or more gear wheels. For example, a single transfer gear wheel would provide that the rotational direction from the outer to the inner dose setting member would be reversed whereas two transfer gear wheels in series would assure rotational movement of the two dose setting members in the same direction. Alternatively, two different-diameter gear wheels may be arranged on a common axis and rotate together, the gear wheels engaging the inner respectively the outer dose setting member.

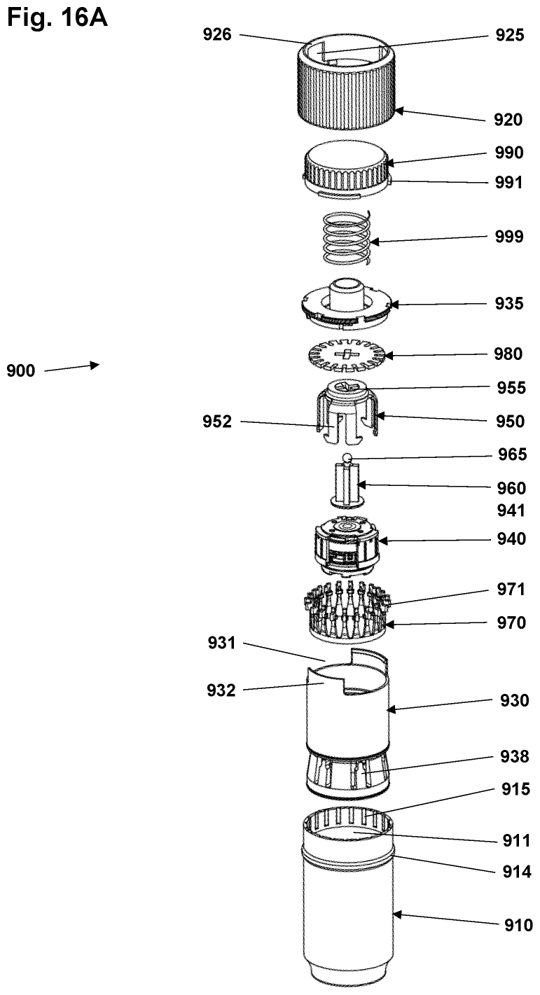

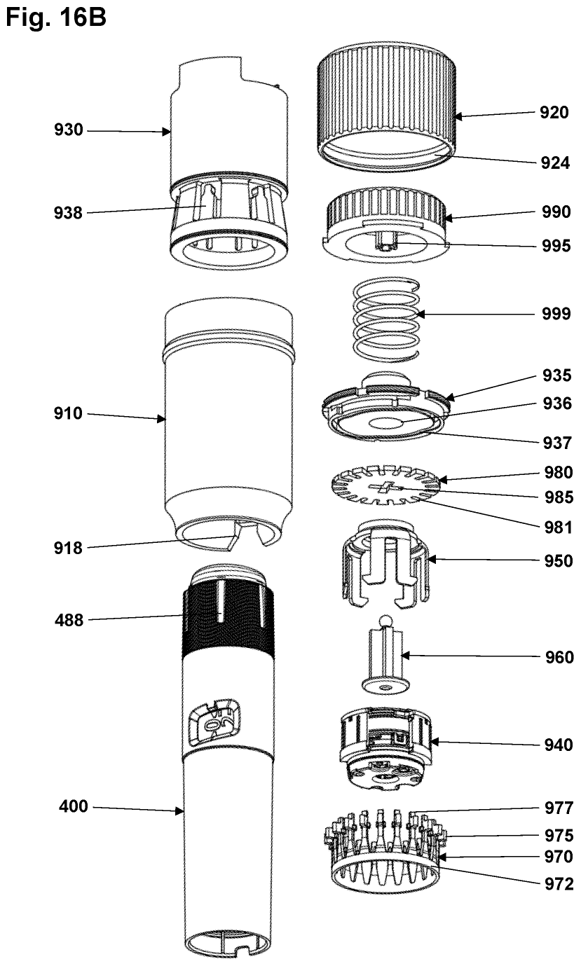

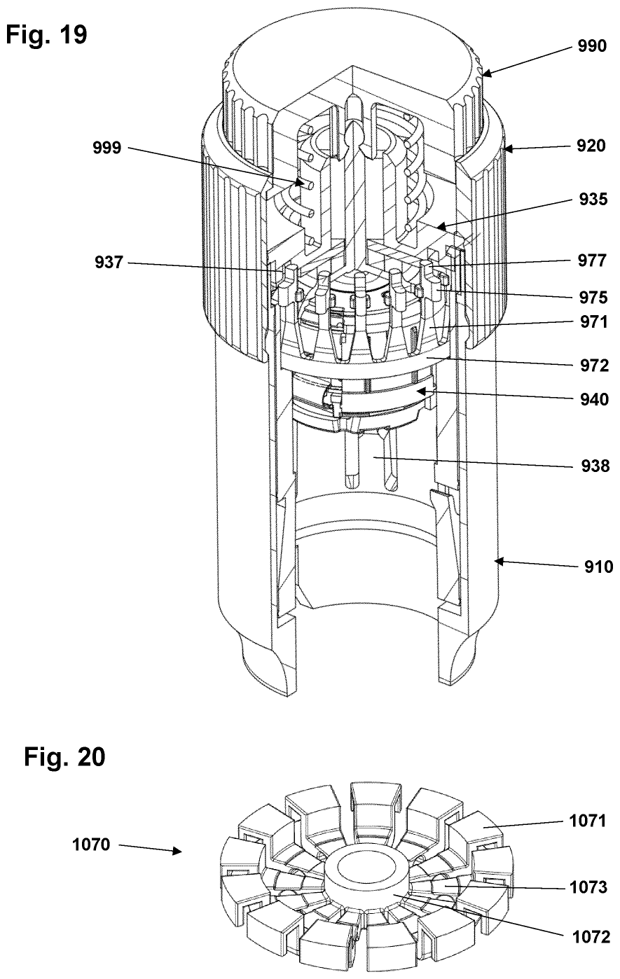

With reference to FIGS. 16A, 16B and 17-19 a third exemplary embodiment of a coupling mechanism allowing an inner and an outer dose setting member to rotate together will be described. Whereas in the first and second exemplary embodiments the concept was to provide a connection through the housing at a level distally of the sensor module, in the third exemplary embodiment the concept is to create a connection between a proximal and a distal portion of the inner dose setting member allowing a rotational input to “pass” the electronic module.

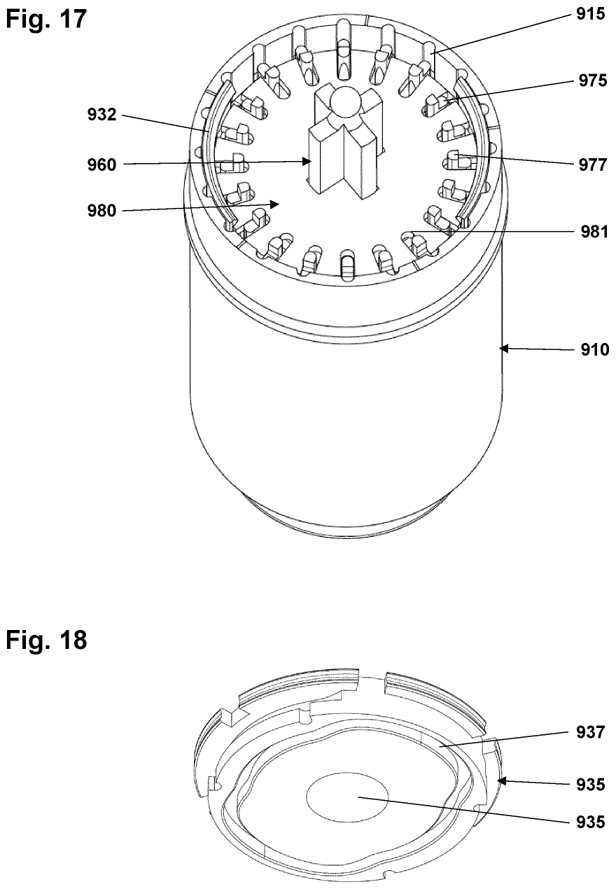

More specifically, the add-on device 900 comprises a tubular housing member 910 with a proximal opening 911 and a distal opening 912 adapted to receive the proximal end of a pen device 400, an outer dose setting 920, an inner tubular dose setting member 930 adapted to engage the pen dose setting member, a sensor module 940 with a proximal switch 941, a module housing 950 adapted to house the sensor module, an actuation rod 960, a finger member 970 with a plurality of flexible fingers 971 adapted to non-rotationally engage the housing, a guide plate 980 non-rotationally coupled to the finger member, a roof member 935, a dose release button 990 axially coupled to the module housing as well as a button return spring 999. FIG. 17 shows in an assembled state the core components providing that the electronic module can be mounted non-rotational in the housing 900.

The housing member 910 comprises an outer circumferential ridge structure 914 at the proximal end adapted to engage the outer dose setting member 920, and an inner circumferential array of axially arranged finger grooves 915 adapted to engage the individual fingers of the finger member 970. The housing member further comprises releasable locking means 918 allowing the add-on device to be mounted axially as well as rotationally locked on the pen housing 400.

The outer dose setting member 920 comprises a generally smooth inner surface adapted to rotate on the housing outer tubular surface, a distal inner circumferential groove 924 adapted to rotationally engage the housing outer ridge structure, guiding tracks 925 for non-rotational engagement with the inner dose setting member 930 (via roof member 935), the guiding tracks comprising a proximal ridge 926 adapted to provide an axial stop for the button member 990.

The tubular inner dose setting member 930 comprises a pair of opposed part-circumferential finger openings 931 dividing the inner dose setting member in a proximal and a distal portion (see below), the two portions being connected by bridge portions 932 between the openings. The proximal portion is in the form of roof member 935 adapted to connect to the distal portion via the bridge portions, the roof member comprising a central opening 936 and a distally facing guide track 937 for the fingers 971 of the finger member 970. The guide track and its functional relationship with the finger member will be described in greater detail below. The distal portion comprises a number of inwardly protruding flexible coupling arms 938 adapted to non-rotationally engage corresponding coupling structures 488 on the pen dose setting member 480. The inner dose setting member is adapted to be mounted axially locked but rotationally free in the housing member 910.

The module housing 950 comprises a proximal non-circular central opening 955 adapted to receive the actuation rod 970 and a number of distally protruding rib structures 952 providing a cage structure adapted to receive and hold the sensor module 940 rotationally locked. The actuation rod 960 has a non-circular cross section allowing it to be non-rotationally mounted through the correspondingly formed opening 955 in the module housing, as well as proximal coupling means 965 (here: a ball coupling) for axially locked engagement with the dose release button 990. The module housing is axially moveable between a proximal and a distal position relative to the inner dose setting member. The actuation rod 960 is allowed to move axially a small amount relative to the cage structure to allow the module switch to be actuated.

The sensor module 940 has a generally cylindrical configuration and is adapted to be mounted non-rotationally in the module housing cage structure. In a situation of use the distal surface of the sensor module is adapted to engage the pen release button 490, this allowing the sensor module switch 941 to be actuated (see below). In the shown embodiment the switch is in the form of a dome switch providing a biasing force on the actuation rod.

The finger member 970 comprises a ring portion 972 with a plurality of proximally extending flexible fingers 971, each finger comprising a lateral protrusion 975 adapted to be received in a corresponding housing finger groove 915 as well as a free end 977 adapted to be received in the roof member guide track 937.

The guide plate 980 comprises a plurality of outer radial guide slots 981 each adapted to receive and guide a corresponding flexible finger 971. In the shown embodiment the guide plate is axially supported on inner protrusions on the individual fingers. The guide plate further comprises a central opening 985 configured to non-rotationally receive the actuation rod 960.

The dose release button 990 comprises circumferential flange portions 991 adapted to engage the guide tracks 925 provided by the outer dose setting member. A distal stop surface is provided by the roof member. The dose release button further comprises a ball coupling socket 995 for engagement with the actuation rod ball coupling 965, this allowing bi-directional axial forces and thus movement to be transmitted to the electronic module. A button return spring 999 ensures that the dose release button is biased towards its proximal position.

Having described the individual components and their relationship of a second exemplary embodiment of an add-on module, a more detailed description of the functionality will be given.

A central component for the second embodiment is the finger element 970 which ensures that the sensor module 940 can be non-rotationally coupled to the housing member 910. Essentially, the finger member is non-rotationally coupled to the housing by means of a plurality of fingers 971, 975 engaging the housing finger grooves 915. However, at the same time the rotational inner dose setting member 930 is arranged between the fingers and the housing member.

To allow such a contradicting arrangement, a pair of opposed “dynamic gaps” are created between the finger member and the housing member. More specifically and as shown in FIG. 17, in a given rotational position of the finger member only a portion of the fingers, e.g. 50%, are in engagement with the finger grooves through the finger openings 931 in the inner dose setting member 930, whereas corresponding to the bridge portions 932 the fingers have been moved radially inwards and out of engagement with the finger grooves, this allowing the bridge portions to pass the finger member. As described above, the free proximal ends 977 of the individual fingers are received in the guide track 937 of the inner dose setting member roof portion, the guide track being designed such that corresponding to the bridge portions the flexible fingers 971 are moved inwardly and out of engagement with the housing finger grooves 915. As the guide track and bridge portions rotate together (being structures of functionally the same component) it is ensured that the fingers are moved inwards allowing the bridge portions to pass and subsequently are moved outwards and into engagement with the housing finger grooves again when the bridge portions have passed. The guide plate slots 981 ensure that the fingers are moved essentially only radially in and out.

In this way it is provided that a constant number of fingers, e.g. 50%, are in engagement with the housing member at all time, this ensuring that the finger member (and thus the electronic module) is “dynamically” mounted non-rotational in the housing member.

In a situation of use the user mounts the add-on device 900 in locking engagement on the proximal end of a corresponding pen drug delivery device 400, whereby the pen dose setting member 480 is received in the inner dose setting member 930. Depending on the design of the two members they may become rotationally locked during the mounting operation or, as in the present embodiment, subsequently when the user starts to set a dose.

To set a dose the user rotates the outer add-on dose setting member 920, and thereby also the inner dose setting member, until the desired dose size is shown in the pen display window. As described above, during dose setting the individual fingers 971 of the finger member will disengage and reengage, this allowing the inner dose setting member bridge portions 932 to pass. During initial rotation of the inner dose setting member the flexible coupling arms 938 will slide on the pen dose setting member until the arms non-rotationally engage corresponding axially oriented coupling grooves 488 on the pen dose setting member.

When a desired dose is set the user actuates dose release button 990 this moving the sensor module 940 into engagement with the pen release button 490. The latter is biased towards its proximal position by a pen button spring, the dome switch 941 being dimensioned to allow the sensor switch to be actuated and thus turn on the sensor module before the pen release button starts to move distally and subsequently releases the spring-loaded expelling mechanism. During dose expelling the sensor module will determine the amount of rotation of an indicator element, e.g. a magnet as in the present example, and thereby the expelled dose.

When a set dose is expelled, or the user desires to pause injection, the user releases pressure on the dose release button, the button return spring 999 thereby returning the dose release button 990 with the sensor module to its initial position.

In FIG. 20 an alternative configuration of a finger member 1070 is shown. Whereas the above-described finger member 970 had a plurality of radially flexible fingers, the alternative configuration comprises a number of axially moveable fingers 1071 connected to a central hub 1072 via a flexible hinge 1074. When implemented in a concrete add-on device the housing structure engaging and disengaging the fingers just as the guide track would be adapted for axial movement instead of radial movement as in the above-described third exemplary embodiment of an add-on logging device.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. An add-on device adapted to be releasably mounted on a drug delivery device, the drug delivery device comprising:
a device housing,
a drug reservoir or structure for receiving a drug reservoir,
drug expelling structure comprising a rotatable dose setting member allowing a user to set a dose amount of drug to be expelled,
a proximally arranged release member actuatable between a proximal position and a distal position, the proximal position allowing a dose amount to be set, the distal position allowing the drug expelling structure to expel a set dose, and
an indicator adapted to move during expelling of a dose amount, the amount of movement being indicative of the size of the expelled dose amount,
the add-on device comprising:
an add-on dose release assembly, and
an add-on dose setting assembly,
the add-on dose release assembly comprising:
a generally tubular add-on housing defining a reference axis, the add-on housing comprising a distal opening adapted to receive the proximal portion of the drug delivery device and being releasably attachable to the drug delivery device housing in a rotationally and axially locked position relative to the device housing, the add-on housing comprising an interior space and an outer surface, and
a sensor assembly arranged in the interior of the tubular add-on housing and coupled, directly or indirectly, non-rotationally yet axially moveable thereto, the sensor assembly being operatable to detect the amount of rotation of the indicator during expelling of a dose amount,
the add-on dose setting assembly comprising:
a gripping member rotationally arranged in the interior of the add-on housing and adapted to non-rotationally engage the dose setting member, and
an add-on dose setting member rotationally coupled to the tubular housing outer surface,
wherein:
the add-on dose setting member is coupled to the gripping member allowing the gripping member to be rotated at least 360 degrees, and
the sensor assembly is axially moveable between a proximal position and a distal position relative to the add-on housing to, with the add-on device mounted on the drug delivery device, engage and actuate the release member when moved distally.

2. An add-on device as in claim 1, wherein:
the add-on housing comprises one or more lateral openings,
the gripping member comprises a circumferential outer gear wheel portion having a first diameter,
the add-on dose setting member comprises a circumferential inner gear wheel portion having a second larger diameter, and
the circumferential outer gear wheel portion and the circumferential inner gear wheel portion engage each other, directly or indirectly, corresponding to a lateral opening,
whereby rotation of the dose setting member provides rotation of the gripping member.

3. An add-on device as in claim 2, wherein:
the rotational axis of the dose setting member is radially offset from the rotational axis of the gripping member, and
the circumferential outer gear wheel portion and the circumferential inner gear wheel portion engage each other corresponding to a lateral opening to form an internal spur gear.

4. An add-on device as in claim 2, further comprising:
a transfer gear assembly comprising at least one gear wheel arranged corresponding to a lateral opening,
wherein the circumferential outer gear wheel portion and the circumferential inner gear wheel portion engage each other via the transfer gear assembly.

5. An add-on device as in claim 2, further comprising:
a circumferential flexible cam belt arranged through a lateral opening and comprising:
an inner toothed surface arranged to engage the gripping member outer gear wheel portion, and
an outer toothed surface arranged to engage the add-on dose setting member inner gear wheel portion,
wherein the flexible cam belt is held in engagement with the gripping member in one or more first positions and in contact with the add-on dose setting member in one or more second positions,
whereby rotation of the add-on dose setting member is transferred to the gripping member via the flexible cam belt.

6. An add-on device as in claim 1, wherein:
the add-on dose release assembly comprises a crown member arranged in the interior of the add-on housing,
the crown member comprises a plurality of coupling fingers adapted to non-rotationally engage the add-on housing, each finger having an engaged state in engagement with the add-on housing and a non-engaged state,
the gripping member comprises at least one bridge portion adapted to pass between the add-on housing and one or more coupling fingers in their non-engaged state, the gripping member being non-rotationally coupled to the add-on dose setting member via the bridge portion(s), and
a control structure coupled non-rotationally to the add-on dose setting member and in engagement with the coupling fingers to move the coupling fingers between the engaged and non-engaged state to thereby allow the bridge portion(s) to pass the coupling fingers as the control structure and the gripping member rotate relative to the crown member.

7. An add-on device as in claim 6, wherein:
the coupling fingers extend axially and are adapted to move radially between the engaged and the non-engaged state, and
the add-on housing comprises a plurality of receiving structures each adapted to receive a given coupling finger in coupling engagement.

8. An add-on device as in claim 7, wherein:
the control structure comprises a circumferential guide track, and
each coupling finger comprises a free end received in the guide track,
whereby the coupling fingers are moved radially in and out of engagement with the add-on housing when the control structure is rotating together with the add-on dose setting member.

9. An add-on device as in claim 8, wherein:
the control structure is arranged proximally of and connected to the bridge portion(s), the guide track facing distally.

10. An add-on device as in claim 1, wherein:
the add-on dose setting member comprises a circumferential first toothing, and
the gripping member comprises a circumferential second toothing, the add-on device further comprising:
a transfer ring arranged at an inclined angle relative to a plane perpendicular to the reference axis, comprising:
(i) a circumferential first transfer toothing in engagement with the add-on dose setting member first toothing, and
(ii) a circumferential second transfer toothing in engagement with the gripping member second toothing,
whereby rotation of the dose setting member provides rotation of the gripping member via the inclined transfer ring.

11. An add-on device in combination with a drug delivery device as defined in claim 1.

12. An add-on device as in claim 1, wherein the add-on dose release assembly further comprises:
a proximally facing user actuatable add-on release member coupled to the sensor assembly and moving axially therewith.

13. A unitary drug delivery device, comprising:
a housing defining a reference axis,
a drug reservoir or structure for receiving a drug reservoir,
drug expelling structure comprising:
an inner dose setting member adapted to rotate to set a dose,
an inner release member actuatable between a proximal position and a distal position, the proximal position allowing a dose amount to be set, the distal position allowing the drug expelling structure to expel a set dose, and
an indicator adapted to rotate during expelling of a dose amount, the amount of rotation being indicative of the size of the expelled dose amount,
a sensor assembly arranged in the interior of the housing and coupled, directly or indirectly, non-rotationally yet axially moveable thereto, the sensor assembly being operatable to detect the amount of rotation of the indicator during expelling of a dose amount, and
a user dose setting member coupled to the housing and adapted to rotate to set a dose,
wherein:
the user dose setting member is rotationally coupled to the inner dose setting member allowing the inner dose setting member to be rotated at least 360 degrees, and
the sensor assembly is axially moveable by the user between a proximal position and a distal position relative to the housing to engage and actuate the inner release member when moved distally.

14. A unitary drug delivery device as in claim 13, wherein:
the housing comprises one or more lateral openings,
the inner dose setting member comprises a circumferential outer gear wheel portion having a first diameter,
the user dose setting member comprises a circumferential inner gear wheel portion having a second larger diameter, and
the circumferential outer gear wheel portion and the circumferential inner gear wheel portion engage each other, directly or indirectly, corresponding to a lateral opening,
whereby rotation of the dose setting member provides rotation of the gripping member.

15. A unitary drug delivery device as in claim 13, further comprising:
a crown member arranged in the interior of the housing, and
a control structure,
wherein:
the crown member comprises a plurality of coupling fingers adapted to non-rotationally engage the housing, each finger having an engaged state in engagement with the add-on housing and a non-engaged state,
the inner dose setting member comprises at least one bridge portion adapted to pass between the housing and one or more coupling fingers in their non-engaged state, the inner dose setting member being non-rotationally coupled to the user dose setting member via the bridge portion(s), and
the control structure is coupled non-rotationally to the user dose setting member and in engagement with the coupling fingers to control the coupling fingers between the engaged and non-engaged state to thereby allow the bridge portion(s) to pass the coupling fingers as the control structure and the inner dose setting member rotate relative to the crown member.
